(19) United States
(12) Patent Application Publication
(10) Pub. No.: US 2003/0150154 A1
(43) Pub. Date: Aug. 14, 2003

(54) Polyalkylphenoxyaminoalkanes and fuel compositions containing the same

(76) Inventor: Richard E. Cherpeck, Cotati, CA (US)

Correspondence Address: Steven G. K. Lee, Chevron Texaco Corporation, P.O. Box 6006, San Ramon, CA, 94583-0806, US

(21) Appl. No.: 10/032,266

(22) Filed: Dec. 21, 2001

(51) Int. Cl. C10L 1/18; C10L 1/22; C07C 215/46

(52) U.S. Cl. 44/424; 564/348

(57) ABSTRACT

Polyalkylphenoxyaminoalkanes having the formula:

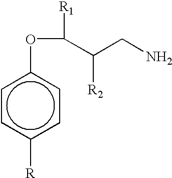

and fuel-soluble salts thereof, wherein

R is a polyalkyl group having an average molecular weight in the range of about 600 to 5,000; and

R₁ and R₂ are independently hydrogen or lower alkyl having about 1 to 6 carbon atoms.

The compounds of the present invention are useful as fuel additives for the prevention and control of engine deposits.

### BACKGROUND OF THE INVENTION

1. Field of the Invention

This invention relates to novel polyalkylphenoxyaminoalkanes. In a further aspect, this invention relates to the use of these compounds in fuel compositions to prevent and control engine deposits.

2. Description of the Related Art

It is well known that automobile engines tend to form deposits on the surface of engine components, such as carburetor ports, throttle bodies, fuel injectors, intake ports and intake valves, due to the oxidation and polymerization of hydrocarbon fuel. These deposits, even when present in relatively minor amounts, often cause noticeable driveability problems, such as stalling and poor acceleration. Moreover, engine deposits can significantly increase an automobile's fuel consumption and production of exhaust pollutants. Therefore, the development of effective fuel detergents or “deposit control” additives to prevent or control such deposits is of considerable importance and numerous such materials are known in the art.

For example, aliphatic hydrocarbon-substituted phenols are known to reduce engine deposits when used in fuel compositions. U.S. Pat. No. 3,849,085, issued Nov. 19, 1974 to Kreuz et al., discloses a motor fuel composition comprising a mixture of hydrocarbons in the gasoline boiling range containing about 0.01 to 0.25 volume percent of a high molecular weight aliphatic hydrocarbon-substituted phenol in which the aliphatic hydrocarbon radical has an average molecular weight in the range of about 500 to 3,500. This patent teaches that gasoline compositions containing minor amounts of an aliphatic hydrocarbon-substituted phenol not only prevent or inhibit the formation of intake valve and port deposits in a gasoline engine, but also enhance the performance of the fuel composition in engines designed to operate at higher operating temperatures with a minimum of decomposition and deposit formation in the manifold of the engine.

U.S. Pat. No. 4,259,086, issued Mar. 31, 1981 to Machleder et al., discloses a detergent additive for fuels and lubricating oils which comprises the reaction product of an aliphatic hydrocarbon-substituted phenol, epichlorohydrin and a primary or secondary monoamine or polyamine. In addition, U.S. Pat. No. 4,048,081, issued Sep. 13, 1977 to Machleder et al., discloses a detergent additive for gasoline which is the reaction product of a polyisobutene phenol with epichlorohydrin, followed by amination with ethylene diamine or other polyamine.

Similarly, U.S. Pat. No. 4,134,846, issued Jan. 16, 1979 to Machleder et al., discloses a fuel additive composition comprising a mixture of (1) the reaction product of an aliphatic hydrocarbon-substituted phenol, epichlorohydrin and a primary or secondary mono- or polyamine, and (2) a polyalkylene phenol. This patent teaches that such compositions show excellent carburetor, induction system and combustion chamber detergency and, in addition, provide effective rust inhibition when used in hydrocarbon fuels at low concentrations.

Amino phenols are also known to function as detergents/dispersants, antioxidants and anti-corrosion agents when used in fuel compositions. U.S. Pat. No. 4,320,021, issued Mar. 16, 1982 to R. M. Lange, for example, discloses amino phenols having at least one substantially saturated hydrocarbon-based substituent of at least 30 carbon atoms. The amino phenols of this patent are taught to impart useful and desirable properties to oil-based lubricants and normally liquid fuels.

In addition, polybutlyamines have been taught to be useful for preventing deposits in the intake system of internal combustion engines. For example, U.S. Pat. No. 4,832,702, issued May 23, 1989 to Kummer et al., discloses fuel and lubricant compositions containing polybutyl or polyisobutylamine additives prepared by hydroformulating a polybutene or polyisobutene and then subjecting the resulting oxo product to a Mannich reaction or amination under hydrogenating conditions.

Polyether amine fuel additives are also well known in the art for the prevention and control of engine deposits. These polyether additives have a polyoxyalkylene “backbone”, i.e., the polyether portion of the molecule consists of repeating oxyalkylene units. U.S. Pat. No. 4,191,537, issued Mar. 4, 1980 to Lewis et al., for example, discloses a fuel composition comprising a major portion of hydrocarbons boiling in the gasoline range and from 30 to 2,000 ppm of a hydrocarbyl polyoxyalkylene aminocarbamate having a molecular weight from about 600 to 10,000, and at least one basic nitrogen atom. The hydrocarbyl polyoxyalkylene moiety is composed of oxyalkylene units having from 2 to 5 carbon atoms in each oxyalkylene unit. These fuel compositions are taught to maintain the cleanliness of intake systems without contributing to combustion chamber deposits.

Aromatic compounds containing a poly(oxyalkylene) moiety are also known in the art. For example, the above-mentioned U.S. Pat. No. 4,191,537, discloses alkylphenyl poly(oxyalkylene) polymers which are useful as intermediates in the preparation of alkylphenyl poly(oxyalkylene) aminocarbamates.

Similarly, U.S. Pat. No. 4,881,945, issued Nov. 21, 1989 to Buckley, discloses a fuel composition comprising a hydrocarbon boiling in the gasoline or diesel range and from about 30 to about 5,000 parts per million of a fuel soluble alkylphenyl polyoxyalkylene aminocarbamate having at least one basic nitrogen and an average molecular weight of about 800 to 6,000 and wherein the alkyl group contains at least 40 carbon atoms.

U.S. Pat. No. 5,112,364, issued May 12, 1992 to Rath et al., discloses gasoline-engine fuels which contain small amounts of a polyetheramine and/or a polyetheramine derivative, wherein the polyetheramine is prepared by reductive amination of a phenol-initiated or alkylphenol-initiated polyether alcohol with ammonia or a primary amine.

European Patent Application Publication No. 310,875, published Apr. 12, 1989 discloses fuels for spark ignition engines containing a polyetheramine additive prepared by first propoxylating and/or butoxylating an alkanol or primary or secondary alkylmonoamine and then aminating the resulting polyether with ammonia or a primary aliphatic amine.

French Patent No. 2,105,539, published Apr. 28, 1972, discloses carburetor detergent additives which are phenoxypropylamines which may be substituted with up to five hydrocarbon radicals of 1 to 30 carbon atoms on the aromatic ring. This patent also discloses additives obtained by reacting such phenoxypropylamines with alkylphosphoric acids.

U.S. Pat. No. 5,669,939 discloses polyalkylphenoxyaminoalkanes as useful fuel additives for the prevention and control of engine deposits, particularly intake valve deposits.

### SUMMARY OF THE INVENTION

The present invention relates to novel polyalkylphenoxyaminoalkanes which provide excellent control of engine deposits, especially intake valve deposits, when employed as fuel additives in fuel compositions.

The compounds of the present invention include those having the following formula:

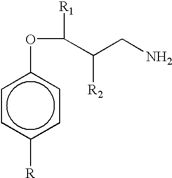

and fuel-soluble salts thereof, wherein

R is a polyalkyl group having an average molecular weight in the range of about 600 to 5,000;

R₁ and R₂ are independently hydrogen or lower alkyl having about 1 to 6 carbon atoms.

The present invention further provides a fuel composition comprising a major amount of hydrocarbons boiling in the gasoline or diesel range and a deposit-controlling effective amount of a compound of the present invention.

The present invention additionally provides a fuel concentrate comprising an inert stable oleophilic organic solvent boiling in the range of from about 150° F. to 400° F. and from about 10 to 70 weight percent of a compound of the present invention.

Among other factors, the present invention is based on the surprising discovery that certain polyalkylphenoxyaminoalkanes provide excellent control of engine deposits, especially on intake valves, when employed as additives in fuel compositions.

### DETAIL DESCRIPTION OF THE INVENTION

The polyalkylphenoxyaminoalkanes of the present invention have the general formula:

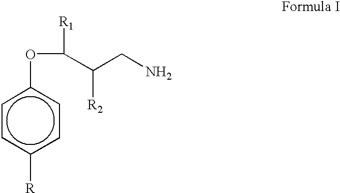

and fuel-soluble salts thereof, wherein R, R₁, and R₂ are as defined herein.

Preferably, R is a polyalkyl group having an average molecular weight in the range of about 600 to 3,000, more preferably about 700 to 3,000, and most preferably about 900 to 2,500.

Preferably, one of R₁ and R₂ is hydrogen or lower alkyl of about 1 to 4 carbon atoms, and the other is hydrogen. More preferably, R₁ is hydrogen and R₂ is hydrogen, methyl or ethyl. Most preferably, both R₁ and R₂ are hydrogen.

It is preferred that the R substituent is located at the meta or, more preferably, the para position on the aromatic ring, i.e., para or meta relative to the ether group.

The compounds of the present invention will generally have a sufficient molecular weight so as to be non-volatile at normal engine intake valve operating temperatures (about 200° C. to 250° C.). Typically, the molecular weight of the compounds of this invention will range from about 700 to about 3,500, preferably from about 700 to about 2,500.

Fuel-soluble salts of the compounds of Formula I can be readily prepared by procedures apparent to those skilled in the art and such salts are contemplated to be useful for preventing or controlling engine deposits. Suitable salts include, for example, those obtained by protonating the amino moiety with a strong organic acid, such as an alkyl- or arylsulfonic acid. Preferred salts are derived from toluenesulfonic acid and methanesulfonic acid.

### Definitions

As used herein, the following terms have the following meanings unless expressly stated to the contrary.

The term “cyano” refers to the group: —CN.

The term “amino” refers to the group: —NH₂.

The term “hydrocarbyl” refers to an organic radical primarily composed of carbon and hydrogen which may be aliphatic, alicyclic, aromatic or combinations thereof, e.g., aralkyl or alkaryl. Such hydrocarbyl groups are generally free of aliphatic unsaturation, i.e., olefinic or acetylenic unsaturation, but may contain minor amounts of heteroatoms, such as oxygen or nitrogen, or halogens, such as chlorine.

The term “alkyl” refers to both straight- and branched-chain alkyl groups.

The term “lower alkyl” refers to alkyl groups having about 1 to 6 carbon atoms and includes primary, secondary and tertiary alkyl groups. Typical lower alkyl groups include, for example, methyl, ethyl, n-propyl, isopropyl, n-butyl, sec-butyl, t-butyl, n-pentyl, n-hexyl and the like.

The term “polyalkyl” refers to an alkyl group which is generally derived from polyolefins which are polymers or copolymers of mono-olefins, particularly 1-mono-olefins, such as ethylene, propylene, butylene, and the like. Preferably, the mono-olefin employed will have about 2 to 24 carbon atoms, and more preferably, about 3 to 12 carbon atoms. More preferred mono-olefins include propylene, butylene, particularly isobutylene, 1-octene and 1-decene. Polyolefins prepared from such mono-olefins include polypropylene, polybutene, especially polyisobutene, and the polyalphaolefins produced from 1-octene and 1-decene.

The term “fuel” or “hydrocarbon fuel” refers to normally liquid hydrocarbons having boiling points in the range of gasoline and diesel fuels.

### General Synthetic Procedures

The polyalkylphenoxyaminoalkanes of this invention may be prepared by the following general methods and procedures. It should be appreciated that where typical or preferred process conditions (e.g., reaction temperatures, times, mole ratios of reactants, solvents, pressures, etc.) are given, other process conditions may also be used unless otherwise stated. Optimum reaction conditions may vary with the particular reactants or solvents used, but such conditions can be determined by one skilled in the art by routine optimization procedures.

Those skilled in the art will also recognize that it may be necessary to block or protect certain functional groups while conducting the following synthetic procedures. In such cases, the protecting group will serve to protect the functional group from undesired reactions or to block its undesired reaction with other functional groups or with the reagents used to carry out the desired chemical transformations. The proper choice of a protecting group for a particular functional group will be readily apparent to one skilled in the art. Various protecting groups and their introduction and removal are described, for example, in T. W. Greene and P. G. M. Wuts, Protective Groups in Organic Synthesis, Second Edition, Wiley, N.Y., 1991, and references cited therein.

### Synthesis

The polyalkylphenoxyaminoalkanes of the present invention may be prepared by a process which initially involves reaction of a polyalkylphenol of the formula:

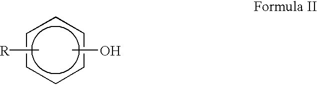

wherein R is as defined herein, with an acrylonitrile of the formula:

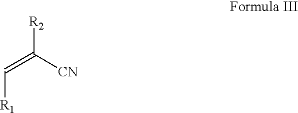

wherein R₁ and R₂ are as defined herein and CN is a cyano group, in the presence of a base to provide the compound of the formula:

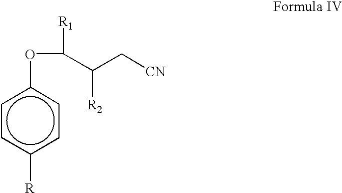

wherein R, R₁, R₂ and CN are as defined herein.

The polyalkylphenols of Formula II are well known materials and are typically prepared by the alkylation of phenol with the desired polyolefin. A further discussion of polyalkylphenols can be found, for example, in U.S. Pat. No. 4,744,921 and U.S. Pat. No. 5,300,701.

Accordingly, the polyalkylphenols of Formula II may be prepared from the corresponding olefins by conventional procedures. For example, the polyalkylphenols of Formula II above may be prepared by reacting the appropriate olefin or olefin mixture with phenol in the presence of an alkylating catalyst at a temperature of from about 25° C. to 150° C., and preferably 30° C. to 100° C. either neat or in an essentially inert solvent at atmospheric pressure. A preferred alkylating catalyst is boron trifluoride.

Molar ratios of reactants may be used. Alternatively, molar excesses of phenol can be employed, i.e., about 2 to 3 equivalents of phenol for each equivalent of olefin with unreacted phenol recycled. The latter process maximizes monoalkylphenol. Examples of inert solvents include heptane, benzene, toluene, chlorobenzene and 250 thinner which is a mixture of aromatics, paraffins and naphthenes.

The polyalkyl substituent on the polyalkylphenols employed in the invention is generally derived from polyolefins which are polymers or copolymers of mono-olefins, particularly 1-mono-olefins, such as ethylene, propylene, butylene, and the like. Preferably, the mono-olefin employed will have about 2 to 24 carbon atoms, and more preferably, 3 to 12 carbon atoms. More preferred mono-olefins include propylene, butylene, particularly isobutylene, 1-octene and 1-decene. Polyolefins prepared from such mono-olefins include polypropylene, polybutene, especially polyisobutene, and the polyalphaolefins produced from 1-octene and 1-decene.

The preferred polyisobutenes used to prepare the presently employed polyalkylphenols are polyisobutenes which comprise at least about 20% of the more reactive methylvinylidene isomer, preferably at least 50% and more preferably at least 70%. Suitable polyisobutenes include those prepared using BF₃ catalysts. The preparation of such polyisobutenes in which the methylvinylidene isomer comprises a high percentage of the total composition is described in U.S. Pat. Nos. 4,152,499 and 4,605,808. Such polyisobutenes, known as “reactive” polyisobutenes, yield high molecular weight alcohols in which the hydroxyl group is at or near the end of the hydrocarbon chain. Examples of suitable polyisobutenes having a high alkylvinylidene content include Ultravis 30, a polyisobutene having a number average molecular weight of about 1300 and a methylvinylidene content of about 74%, and Ultravis 10, a polyisobutene having a number average molecular weight of about 950 and a methylvinylidene content of about 76%, both available from British Petroleum.

The acrylonitrile of Formula III are known compounds which are available commercially or can be readily prepared using conventional procedures.

The base employed in the reaction of the polyalkylphenol and acrylonitrile may be any of the well-known catalysts. Typical catalysts include alkali metal hydroxides, alkoxides and hydrides, alkali metal salts, and tetrahydrocarbyl ammonium hydroxides and alkoxides.

The amount of base employed will generally range from about 0.001 to 1.0 equivalent, preferably from about 0.01 to 0.1 equivalent.

The acrylonitrile employed will generally range from about 1 to 20 equivalents, preferably from about 1 to 10 equivalents. The reaction of the polyalkylphenol and acrylonitrile in the presence of the hydroxyalkylation catalyst occurs at a temperature in the range of about 20° C. to 100° C., and preferably from about 25° C. to 65° C. The reaction may take place in the presence or absence of an inert solvent. The time of reaction will vary depending on the particular polyalkylphenol and acrylonitrile reactants, the catalyst used and the reaction temperature.

The CN group of Formula IV may be reduced by any number of procedures well known in the art to a —CH₂NH₂ group under catalytic hydrogenation conditions to yield the polyalkylphenoxyaminoalkane of the present invention (Formula I). Typically, this reaction is conducted using a nickel, Raney nickel, cobalt, Raney cobalt, copper-chromite, platinum, palladium, or rhodium catalyst. Preferably, the catalyst is nickel, Raney nickel, or platinum. The hydrogen pressure, time, and temperature depend on the catalyst employed. An inert solvent may be employed such as ethanol, ethyl acetate, and the like. Ammonium may also be added as a diluent. Hydrogenation of CN groups is further discussed, for example, in P. N. Rylander, Catalytic Hydrogenation in Organic Synthesis, Second Edition, pp.138-152, Academic Press (1979) and H. F. Rase, Handbook of Commercial Catalysts, Heterogeneous Catalyst, pp. 138-148, CRC Press (2000) and references cited therein.

### Fuel Compositions

The compounds of the present invention are useful as additives in hydrocarbon fuels to prevent and control engine deposits, particularly intake valve deposits. The proper concentration of additive necessary to achieve the desired deposit control varies depending upon the type of fuel employed, the type of engine, and the presence of other fuel additives.

In general, the concentration of the compounds of this invention in hydrocarbon fuel will range from about 25 to 5,000 parts per million (ppm) by weight, preferably from 50 to 1,000 ppm. When other deposit control additives are present, a lesser amount of the present additive may be used.

The compounds of the present invention may be formulated as a concentrate using an inert stable oleophilic (i.e., dissolves in gasoline) organic solvent boiling in the range of about 150° F. to 400° F. (about 65° C. to 205° C.). Preferably, an aliphatic or an aromatic hydrocarbon solvent is used, such as benzene, toluene, xylene or higher-boiling aromatics or aromatic thinners. Aliphatic alcohols containing about 3 to 8 carbon atoms, such as isopropanol, isobutylcarbinol, n-butanol and the like, in combination with hydrocarbon solvents are also suitable for use with the present additives. In the concentrate, the amount of the additive will generally range from about 10 to 70 weight percent, preferably 10 to 50 weight percent, more preferably from 20 to 40 weight percent. In gasoline fuels, other fuel additives may be employed with the additives of the present invention, including, for example, oxygenates, such as t-butyl methyl ether, antiknock agents, such as methylcyclopentadienyl manganese tricarbonyl, and other dispersants/detergents, such as hydrocarbyl amines, hydrocarbyl poly(oxyalkylene) amines, hydrocarbyl poly(oxyalkylene) aminocarbamates, Mannichs or succinimides. Additionally, antioxidants, metal deactivators and demulsifiers may be present.

In diesel fuels, other well-known additives can be employed, such as pour point depressants, flow improvers, cetane improvers, and the like.

A fuel-soluble, nonvolatile carrier fluid or oil may also be used with the compounds of this invention. The carrier fluid is a chemically inert hydrocarbon-soluble liquid vehicle which substantially increases the nonvolatile residue (NVR), or solvent-free liquid fraction of the fuel additive composition while not overwhelmingly contributing to octane requirement increase. The carrier fluid may be a natural or synthetic oil, such as mineral oil, refined petroleum oils, synthetic polyalkanes and alkenes, including hydrogenated and unhydrogenated polyalphaolefins, and synthetic polyoxyalkylene-derived oils, such as those described, for example, in U.S. Pat. No. 4,191,537 to Lewis, and polyesters, such as those described, for example, in U.S. Pat. Nos. 3,756,793 to Robinson and 5,004,478 to Vogel et al., and in European Patent Application Nos. 356,726, published Mar. 7, 1990, and 382,159, published Aug. 16, 1990.

These carrier fluids are believed to act as a carrier for the fuel additives of the present invention and to assist in removing and retarding deposits. The carrier fluid may also exhibit synergistic deposit control properties when used in combination with a compound of this invention.

The carrier fluids are typically employed in amounts ranging from about 25 to 5,000 ppm by weight of the hydrocarbon fuel, preferably from 50 to 3,000 ppm of the fuel. Preferably, the ratio of carrier fluid to deposit control additive will range from about 0.5:1 to 10:1, more preferably from 0.5:1 to 3:1, most preferably about 1:1. When employed in a fuel concentrate, carrier fluids will generally be present in amounts ranging from about 20 to 60 weight percent, preferably from 30 to 50 weight percent.

### EXAMPLES

The invention will be further illustrated by the following examples, which set forth particularly advantageous process embodiments. While the Examples are provided to illustrate the present invention, they are not intended to limit it. This application is intended to cover those various changes and substitutions that may be made by those skilled in the art without departing from the spirit and scope of the appended claims.

### EXAMPLE 1

### Preparation of

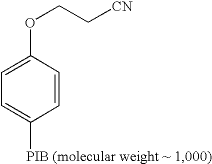

4-Polyisobutyl (molecular weight˜1,000) phenol (21.86 grams, prepared as in Example 1 of U.S. Pat. No. 5,300,701) and toluene (50 mL) were added to a flask equipped with a magnetic stirrer, reflux condenser and nitrogen inlet. Benzyltrimethylammonium methoxide (0.45 grams of a 40 weight percent solution in methanol) was added and the reaction was refluxed for 2 hours. The solvents were distilled off and acrylonitrile (6.6 mL, passed through activated alumina and then distilled) was added. The reaction was heated at 75° C. for sixteen hours. The reaction was cooled to room temperature and the solvents removed in vacuo. The oil was chromatographed on silica gel, eluting with hexane followed by hexane/ethyl acetate (90:10) to yield 19.0 grams of the desired product as a colorless oil. ¹H-NMR (CDCl₃) 7.25 (ABq, 2H), 6.8 (ABq, 2H), 4.2 (t, 2H), 2.8 (t, 2H), 0.7-1.6 (m, 145H).

### EXAMPLE 2

### Preparation of

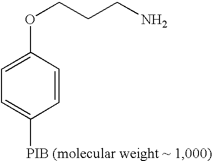

A solution of 19.0 grams of the product from Example 1 in 50 mL of ethyl acetate and 50 mL of toluene containing 2.0 gram of platinum(IV) oxide was hydrogenated at 50 psi for 62 hours on a Parr low-pressure hydrogenator. Catalyst filtration and removal of the solvents in vacuo yielded an oil. The oil was chromatographed on silica gel, eluting with hexane/ethyl acetate (70:30) followed by hexane/diethyl ether/methanol/isopropylamine (40:40:15:5) to afford 5.0 grams of the desired product as an oil. ¹H-NMR (CDCl₃/D₂O) 7.25 (ABq, 2H), 6.8 (ABq, 2H), 4.0 (t, 2H), 2.9 (t, 2H), 1.95 (p, 2H), 0.7-1.5 (m, 145H).

### EXAMPLE 3

### Single-Cylinder Engine Test

The test compounds were blended in gasoline and their deposit reducing capacity determined in an ASTM/CFR single-cylinder engine test.

A Waukesha CFR single-cylinder engine was used. Each run was carried out for 15 hours, at the end of which time the intake valve was removed, washed with hexane and weighed. The previously determined weight of the clean valve was subtracted from the weight of the value at the end of the run. The difference between the two weights is the weight of the deposit. A lesser amount of deposit indicates a superior additive. The operating conditions of the test were as follows: water jacket temperature 200° F.; vacuum of 12 in Hg, air-fuel ratio of 12, ignition spark timing of 400 BTC; engine speed is 1,800 rpm; the crankcase oil is a commercial 30W oil. The amount of carbonaceous deposit in milligrams on the intake valves is reported for each of the test compounds in Table I.

TABLE I

| | Intake Valve Deposit Weight | | |
|---|---|---|---|
| | (in milligrams) | | |
| Sample¹ | Run 1 | Run 2 | Average |
| Base Fuel | 208.2 | 209.8 | 209.0 |
| Example 2 | 10.7 | 12.6 | 11.7 |

^{1 At 30 parts per million actives (ppma).}

The base fuel employed in the above single-cylinder engine tests was a regular octane unleaded gasoline containing no fuel detergent. The test compounds were admixed with the base fuel to give the concentrations indicated in the tables.

The data in Table I illustrates the significant reduction in intake valve deposits provided by the polyalkylphenoxyaminoalkanes of the present invention (Example 2) compared to the base fuel.

What is claimed is:

1. A compound of the formula: 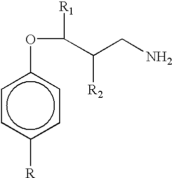
and fuel-soluble salts thereof, wherein
R is a polyalkyl group having an average molecular weight range of about 600 to 5,000; and
R₁ and R₂ are independently hydrogen or lower alkyl having about 1 to 6 carbon atoms.

2. The compound according to claim 1, wherein R is a polyalkyl group having an average molecular weight in the range of about 600 to 3,000.

3. The compound according to claim 2, wherein R is a polyalkyl group having an average molecular weight in the range of about 700 to 3,000.

4. The compound according to claim 3, wherein R is a polyalkyl group having an average molecular weight in the range of about 900 to 2,500.

5. The compound according to claim 1, wherein R is a polyalkyl group derived from polypropylene, polybutene, or a polyalphaolefin oligomer of 1-octene or 1 -decene.

6. The compound according to claim 5, wherein R is a polyalkyl group derived from polyisobutene.

7. The compound according to claim 6, wherein the polyisobutene contains at least about 20% of a methylvinylidene isomer.

8. The compound according to claim 1, wherein one of R₁ and R₂ is hydrogen or lower alkyl of about 1 to 4 carbon atoms, and the other is hydrogen.

9. The compound according to claim 8, wherein R₁ is hydrogen and R₂ is hydrogen, methyl or ethyl.

10. The compound according to claim 9, wherein both R₁ and R₂ are hydrogen.

11. A fuel composition comprising a major amount of hydrocarbons boiling in the gasoline or diesel range and an effective deposit-controlling amount of a compound of the formula: 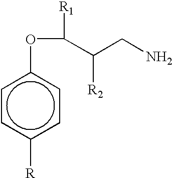
and fuel-soluble salts thereof, wherein
R is a polyalkyl group having an average molecular weight range of about 600 to 5,000; and
R₁ and R₂ are independently hydrogen or lower alkyl having about 1 to 6 carbon atoms.

12. The fuel composition according to claim 11, wherein R is a polyalkyl group having an average molecular weight in the range of about 600 to 3,000.

13. The fuel composition according to claim 12, wherein R is a polyalkyl group having an average molecular weight in the range of about 700 to 3,000.

14. The fuel composition according to claim 13, wherein R is a polyalkyl group having an average molecular weight in the range of about 900 to 2,500.

15. The fuel composition according to claim 11, wherein R is a polyalkyl group derived from polypropylene, polybutene, or a polyalphaolefin oligomer of 1-octene or 1-decene.

16. The fuel composition according to claim 15, wherein R is a polyalkyl group derived from polyisobutene.

17. The fuel composition according to claim 16, wherein the polyisobutene contains at least about 20% of a methylvinylidene isomer.

18. The fuel composition according to claim 11, wherein one of R₁ and R₂ is hydrogen or lower alkyl of about 1 to 4 carbon atoms, and the other is hydrogen.

19. The fuel composition according to claim 18, wherein R₁ is hydrogen and R₂ is hydrogen, methyl or ethyl.

20. The fuel composition according to claim 19, wherein both R₁ and R₂ are hydrogen.

21. The fuel composition according to claim 11, wherein the composition contains from about 25 to 5,000 parts per million by weight of said compound.

22. The fuel composition according to claim 21, wherein the composition contains from about 50 to 1,000 parts per million by weight of said compound.

23. The fuel composition according to claim 11, wherein the composition further contains from about 25 to 5,000 parts per million by weight of a fuel-soluble, nonvolatile carrier fluid.

24. The fuel composition according to claim 23, wherein the composition further contains from about 50 to 3,000 parts per million by weight of a fuel-soluble, nonvolatile carrier fluid.

25. A fuel concentrate comprising an inert stable oleophilic organic solvent boiling in the range of from about 150° F. to 400° F. and from about 10 to 70 weight percent of a compound of the formula: 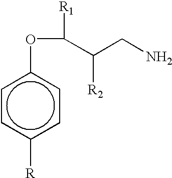
and fuel-soluble salts thereof, wherein
R is a polyalkyl group having an average molecular weight range of about 600 to 5,000; and
R₁ and R₂ are independently hydrogen or lower alkyl having about 1 to 6 carbon atoms.

26. The fuel concentrate according to claim 25, wherein R is a polyalkyl group having an average molecular weight in the range of about 600 to 3,000.

27. The fuel concentrate according to claim 26, wherein R is a polyalkyl group having an average molecular weight in the range of about 700 to 3,000.

28. The fuel concentrate according to claim 27 wherein R is a polyalkyl group having an average molecular weight in the range of about 900 to 2,500.

29. The fuel concentrate according to claim 25, wherein R is a polyalkyl group derived from polypropylene, polybutene, or a polyalphaolefin oligomer of 1-octene or 1-decene.

30. The fuel concentrate according to claim 29, wherein R is a polyalkyl group derived from polyisobutene.

31. The fuel concentrate according to claim 30, wherein the polyisobutene contains at least about 20% of a methylvinylidene isomer.

32. The fuel concentrate according to claim 25, wherein one of R₁ and R₂ is hydrogen or lower alkyl of about 1 to 4 carbon atoms, and the other is hydrogen.

33. The fuel concentrate according to claim 32, wherein R₁ is hydrogen and R₂ is hydrogen, methyl or ethyl.

34. The fuel concentrate according to claim 33 wherein both R₁ and R₂ are hydrogen.

35. The fuel concentrate according to claim 25, wherein the fuel concentrate further contains from about 20 to 60 weight percent of a fuel-soluble, nonvolatile carrier fluid.
